(19) United States
(12) Patent Application Publication
(10) Pub. No.: US 2026/0158280 A1
(43) Pub. Date: Jun. 11, 2026

(54) MULTI-STAGE ATRIAL CARDIOVERSION THERAPY LEADS

(71) Applicant: MAXWELL BIOMEDICAL, INC., San Diego, CA (US)

(72) Inventor: Brent Michael SHELTON, Golden Valley, MN (US)

(21) Appl. No.: 19/269,164

(22) Filed: Jul. 15, 2025

Related U.S. Application Data
(63) Continuation of application No. 18/419,702, filed on Jan. 23, 2024, now abandoned.
Application No. 18/419,702 is a continuation of application No. 17/140,333, filed on Jan. 4, 2021, now Pat. No. 11,918,816.
Application No. 17/140,333 is a continuation of application No. 13/947,840, filed on Jul. 22, 2013, now Pat. No. 10,905,884.
(60) Provisional application No. 61/674,145, filed on Jul. 20, 2012.

(51) Int. Cl. A61N 1/365; A61N 1/362; A61N 1/368

(52) U.S. Cl. CPC A61N 1/36514; A61N 1/3624; A61N 1/3686

(57) ABSTRACT

Devices and methods of use for treating atrial arrhythmias. A single-pass lead includes a body portion having at least two electrodes configured to be positioned within or adjacent a right atrium of a heart of a patient, and a distal portion having at least two electrodes configured to be positioned within a blood vessel proximate the left atrium. The lead is configured to be operated by an implantable therapy generator programmed to deliver a multi-stage therapy by activating various combinations of at least one electrode of the body portion of the lead and at least one electrode of the distal portion of the lead.

### RELATED APPLICATIONS

The present application is a continuation of U.S. patent application Ser. No. 18/419,702, filed Jan. 23, 2024, which is a continuation of U.S. patent application Ser. No. 17/140,333, filed Jan. 4, 2021, which is a continuation of U.S. patent application Ser. No. 13/947,840, filed Jul. 22, 2013, which claims the benefit of U.S. Provisional Patent Application No. 61/674,145, filed Jul. 20, 2012, titled “Multi-Stage Atrial Cardioversion Therapy Leads,” the disclosure of each of which is hereby incorporated by reference.

### TECHNICAL FIELD

The present invention is generally directed to devices, systems and methods for treating atrial fibrillation. More specifically, the present invention is directed to implantable electrical leads for delivering multi-stage, atrial cardioversion therapy to a patient.

### BACKGROUND

Traditional electrical shock therapies for treating an atrial arrhythmia in a patient typically include delivery of a series of mono-or biphasic shocks, each shock, or pulse, often having a similar electrical characteristic. To deliver such traditional therapy, a wide variety of electrical leads may be used. However, for multi-stage electrical shock therapies, each stage having a potentially different electrical characteristic, the use of standard, known leads may be insufficient.

### BRIEF DESCRIPTION OF THE DRAWINGS

The invention may be more completely understood in consideration of the following detailed description of various embodiments of the invention in connection with the accompanying drawings, in which:

FIG. 1 depicts an embodiment of a single-pass multi-stage therapy lead, according to an embodiment of the claimed invention;

FIG. 2 depicts a switchable and selectable lead, according to an embodiment of the claimed invention;

FIGS. 3-6 depict embodiments of leads of the claimed invention as applied to treat an atrial arrhythmia;

FIG. 7 depicts the selection of leads and lead combinations depending on arrhythmia maturity;

FIG. 8 depicts another lead, according to an embodiment of the claimed invention; and

FIG. 9 depicts yet another lead, according to an embodiment of the claimed invention.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION OF THE DRAWINGS

When applying multi-stage therapies, there exists a clinical need to minimize lead hardware and maximize therapy options. Embodiments of the present invention minimize the total number of leads required, thereby increasing ease and speed of an implant, and may reduce the total number of conductors in leads, thereby increasing reliability. Published U.S. Patent Application No. 2012/0209343 to Efimov et al., describes and depicts a number of multi-stage atrial cardioversion therapies, the contents of which, other than the claims and express teachings, are herein incorporated by reference in their entirety. Further, it will be understood that the details and variations of construction of leads in accordance with the various embodiments of the present invention can be accomplished in any number of manners of lead construction known to a person of skill in the art and/or in accordance with conventional or proposed standards for implantable leads such as the International Standard 1 (IS-1), DF-1, and IS-6 standards, the disclosures and details of such standards being incorporated herein by reference.

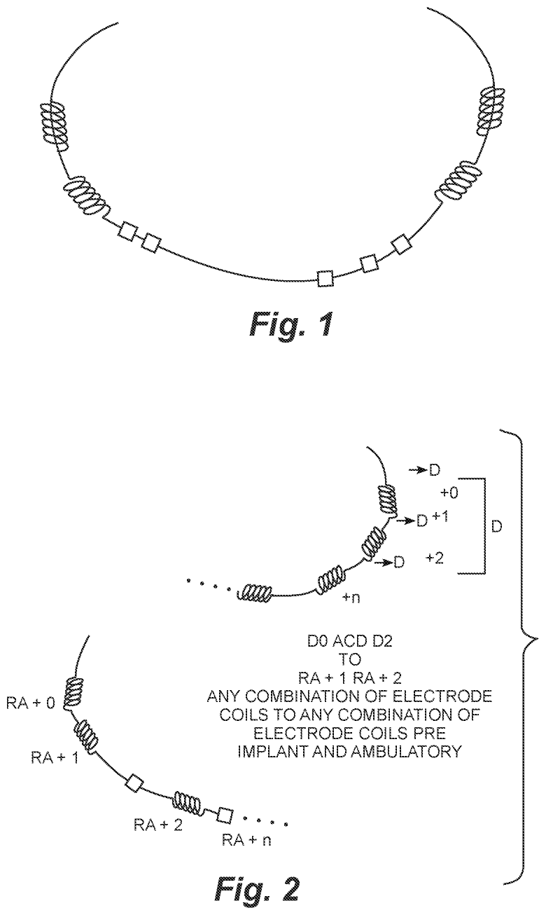

Embodiments of the claimed invention include one or more electrical leads as described below and as depicted in FIGS. 1-9. Embodiments of the claimed leads may incorporate one or more of the concepts and or features described below. Each concept may be included in existing leads and/or combined into a multi-stage therapy (MST) lead, which is a lead specifically designed to reduce complexity and time of implant and maximize therapeutic options for delivery of bi-atrial multi-stage therapy (or simply bi-atrial and/or one or more stage therapy).

In an embodiment, the claimed invention comprises a lead having an electrode configuration having physiologic spacing that permits maximum physiologic response.

Referring to FIG. 1, the lead comprises a plurality of high-voltage coils in the coronary sinus and right atrial superior vera cava. The distance between coils permits or facilitate a single-pass lead. So, these distal coronary sinus coils are a physiologic specific distance, (given other dual or multi-coil lead configurations) between each other. The clinical objective is to maximize the trans-atrial electrical field for any therapy, but also for multi-stage therapy.

In an embodiment, the lead comprises a plurality of pacing electrodes proximal to the MST coil (vs. tip-ring being distal to the coil) both in the left atrial CS to permit bi-atrial or uni-bi-atrial pacing.

In another embodiment, the lead includes a plurality of electrodes in the right atrium (i. e, not in the coronary sinus) and will permit pacing or sensing of electrical activity.

The spacing of the LA (non-descending) coronary sinus electrodes and coils are unique to human physiology to permit the reduction of the number of the total number of leads.

In an embodiment, the lead includes a coil length which is matched to the physiologic need to maximally cover the left atrium without creating a shortened pathway to the right atrial coils. The lead may accommodate a physiologic distance to one or more physiologically positioned coils so as to maximize atrial recruitment.

Referring to FIG. 2, in another embodiment, the lead includes switchable/selectable coil pairs and/or more coils. Embodiments of the claimed invention may be combined with methods and apparatuses for switching and selecting as known by those skilled in the art, such that any combination of electrode coils or any combination of electrode coils, pre-implant and ambulatory, are possible.

Such embodiments of the claimed invention may include the ability to select electrode coil pairs pre-implant, per implant or ambulatory for the purpose to maximize first, second, third, or even nth stage therapy. Some such therapies may be coil-delivered, not pacing-delivered, Rx.

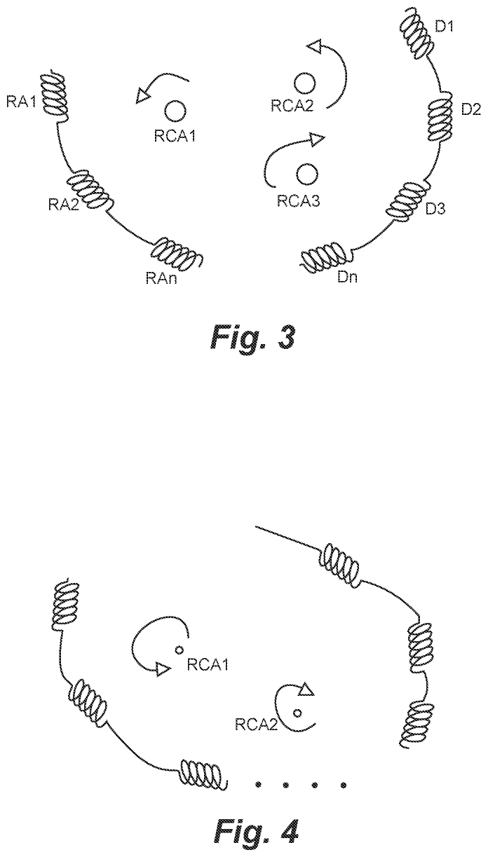

Referring also to FIG. 3, the ability to select electrode coil pairs pre-implant or ambulatory may facilitate the maximization of one or more of a first, second, third, or nth stage therapy/Rx. Such therapies may be coil-delivered or pacing-delivered therapies.

Defibrillatory efficacy or refractory extension prolongation while minimizing required energy: Stage 1, 2, . . . n therapy may be delivered for arranging disruption (unpinning) and refractory period. Prolongation is phase dependent and, thus, position of energy dependent. As depicted in FIG. 3, re-entrant circuit arrhythmia 1, 2 and 3 may be operating in a unique and asynchronous or synchronous fashion (i.e., RCA1 may or may not support RCA2 or RCA3). To maximize the effect of disruption/unpinning and/or refractory period modification: Therapy may be delivered to maximize current density between two or more coils. So, D3 to RA1 may impact RCA3 and RCA1, which may be dominate foci and thus disrupt RCA2's ability to sustain-do not need to reach RCA 1, 2 and 3—just the dominate foci at that time for that arrhythmia. Alternatively, if RCA3 was dominate, RAn to D3 may address RCA3, not reaching RCA1 or RCA2 with energy, but that was not needed, as the lower energy RCA3 pair effectively disrupted the interrelationship to RCA1 and RCA2 indirectly terminating entire arrhythmia.

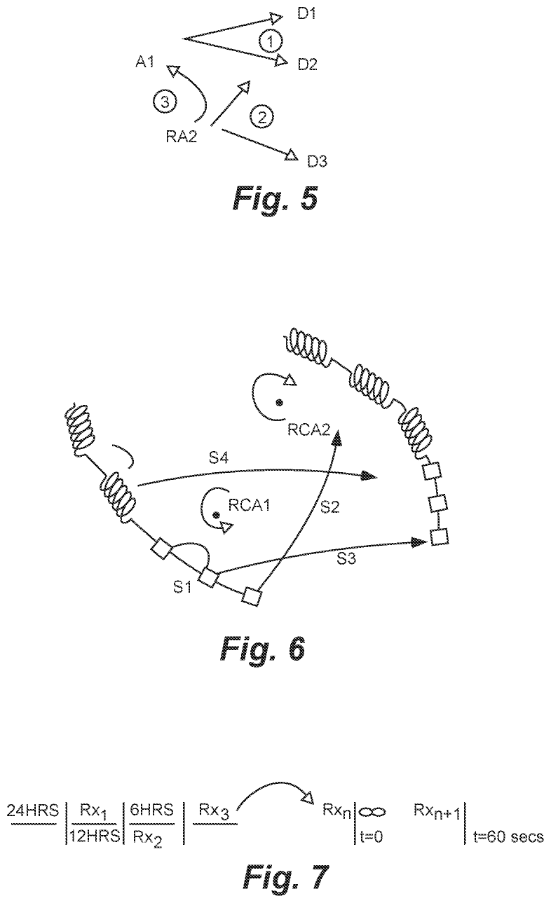

Referring also to FIGS. 4 and 5, in an embodiment, the claimed invention is adapted and configured to find/disrupt the arrhythmia. The system of leads may search to deliver therapy through any number of combinations of coils. The system may search through numerous coil pairs/combination to seek out the presenting arrhythmia because time is not life critical. So, while a total of 1 Joule between any two pair may reliably disrupt an arrhythmia, one would search

$${C}_{m}^{n}$$

combinations of, say, 0.1 J therapies between any number of coil combinations, for example, therapy (1) is between RA1 and D1/D2.

Embodiments of the claimed invention may include a series of these combinations of therapies which first disrupt and then extinguish the arrhythmia. For example, the therapy may require delivery of first combination pathway (2) followed by (3) and then (1) to be successful. The arrhythmia becomes unpinned at, for example, RCA2 only to become established around RCA1, whereby sequence (3) then (2), terminate the arrhythmia.

In an embodiment, the system learns which combination works best to establish which arrhythmia. The sequence of therapies which maximizes termination with minimal energy becomes probabilistically the therapy delivered and/or attempted. The system watches to determine which therapies and therapy combination are most effective.

Referring to FIG. 6, in an embodiment, the system monitors the arrhythmia on any number of combinations of coils and electrodes. Given that any arrhythmia is subject to measurement observation—RCA1 will appear different when obscured on S1, S4, etc.

Referring also to FIG. 7, the treatment of arrhythmias may be dependent not only on the combination of stage therapy, the pair or number of poles (coils and/or electrode combinations) but the maturity of the arrhythmia.

For an arrhythmia of longer duration, a more aggressive therapy (Rx₁) may be required. As noted above, the therapy may be comprised of any number of stages and/or combinations of therapy stages delivered through any number of combinations of coils/electrodes. Given that shorter duration arrhythmias are more easily terminated, the therapy may start with less aggressive and, presumably, less perceptible therapy.

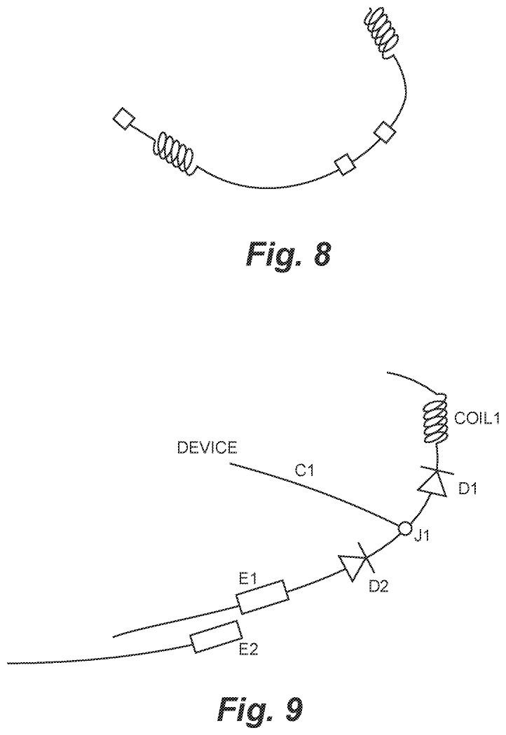

Referring to FIG. 8, a number of pacing electrodes proximal to the distal coil located at a physiologic distance from the coils and electrodes which would permit physiologic B12-atrial pacing from a single-pass MST B1-atrial pacing lead, the electrodes would be selectable. As described elsewhere for 1 to n electrodes, these would be selected to maximize therapy and minimize current drain. The electrodes could be coils.

Any number of selectable electrode configurations have been previously disclosed herein. Here the use of passive electrical devices leverage the relative energy delivered between various coils, electrodes given the relative energy levels of Stage 1 and on. The concept is to minimize the number of unique conductors relative to the current pathways.

Referring also to FIG. 9, the selective use of passive diodes can be used to direct current. For example, fewer conductors can be used knowing different energy will be delivered down differing electrode/coil configurations.

So, in an embodiment, to deliver S1 therapy, conductor C1 is energized through junction J1. Diode D1 is forward biased and diode D2 is reverse biased, preventing current from flooding to electrode E1. The S1 therapy is channeled to coil 1. If pacing therapy is, for example, delivered to E1 with C1 being the return pathway (V=0), current is preferentially directed back to the device through C1. Diode 1 offset voltage say 0.7 v, is not overcome and little/no current flows to coil 1.

Various embodiments of the claimed invention are further described and depicted in the attached document Attachment A, “Multi-Stage Therapy Leads”, comprising 6 pages. Attachment A is incorporated by reference herein in its entirety.

Various embodiments of systems, devices and methods have been described herein. These embodiments are given only by way of example and are not intended to limit the scope of the invention. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the invention.

Persons of ordinary skill in the relevant arts will recognize that the invention may comprise fewer features than illustrated in any individual embodiment described above. The embodiments described herein are not meant to be an exhaustive presentation of the ways in which the various features of the invention may be combined. Accordingly, the embodiments are not mutually exclusive combinations of features; rather, the invention may comprise a combination of different individual features selected from different individual embodiments, as understood by persons of ordinary skill in the art.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims for the present invention, it is expressly intended that the provisions of Section 112, sixth paragraph of 35 U.S.C. are not to be invoked unless the specific terms “means for” or “step for” are recited in a claim.

In one example embodiment, an implantable apparatus is provided for treatment of atrial arrhythmia of a patient using an implantable therapy generator. The apparatus includes: a single-pass lead configured for implantation in the patient such that no electrodes are within a right ventricle of a heart of the patient, the lead including: a proximal portion configured to be connectable to the implantable therapy generator; a body portion having at least two electrodes, the lead configured such that when implanted in the patient the at least two electrodes on the body portion are positioned within or adjacent a right atrium of a heart of a patient; and a distal portion having at least two electrodes, the lead configured such that when implanted in the patient the at least two electrodes on the distal portion are positioned within a blood vessel proximate the left atrium, wherein the implantable therapy generator is programmed to deliver a multi-stage therapy by activating various combinations of at least one electrode of the body portion of the lead and at least one electrode of the distal portion of the lead in response to an indication an atrial arrhythmia has occurred, the multi-stage therapy including at least a first stage for unpinning of one or more singularities associated with the atrial arrhythmia, a second stage for anti-repinning of the one or more singularities associated with the atrial arrhythmia, and a third stage for extinguishing of the one or more singularities associated with the atrial arrhythmia.

In another example embodiment, a method includes: providing a single-pass lead configured for implantation in a patient, the lead including: a proximal portion configured to be connectable to the implantable therapy generator; a body portion having at least two electrodes; and a distal portion having at least two electrodes; and providing instructions, the instructions comprising: implanting the single-pass lead in the patient such that the at least two electrodes on the body portion are positioned within or adjacent a right atrium of a heart of a patient, and such that the at least two electrodes on the distal portion are positioned within a blood vessel proximate the left atrium, and further such that no electrodes are within a right ventricle of a heart of the patient; and causing the implantable therapy generator to deliver a multi-stage therapy according to a program, wherein various combinations of at least one electrode of the body portion of the lead and at least one electrode of the distal portion of the lead are activated in response to an indication an atrial arrhythmia has occurred, the multi-stage therapy including at least a first stage for unpinning of one or more singularities associated with the atrial arrhythmia, a second stage for anti-repinning of the one or more singularities associated with the atrial arrhythmia, and a third stage for extinguishing of the one or more singularities associated with the atrial arrhythmia, wherein each of the stages includes a plurality of pulses.

In an additional example embodiment, a method is provided for treating atrial arrhythmias in a patient with a single-pass lead, the lead including a proximal portion configured to be connectable to the implantable therapy generator, a body portion having at least two electrodes, and a distal portion having at least two electrodes. The method includes: implanting the single-pass lead in the patient such that the at least two electrodes on the body portion are positioned within or adjacent a right atrium of a heart of a patient, and such that the at least two electrodes on the distal portion are positioned within a blood vessel proximate the left atrium, and further such that no electrodes are within a right ventricle of a heart of the patient; and causing the implantable therapy generator to deliver a multi-stage therapy according to a program, wherein various combinations of at least one electrode of the body portion of the lead and at least one electrode of the distal portion of the lead are activated in response to an indication an atrial arrhythmia has occurred, the multi-stage therapy including at least a first stage for unpinning of one or more singularities associated with the atrial arrhythmia, a second stage for anti-repinning of the one or more singularities associated with the atrial arrhythmia, and a third stage for extinguishing of the one or more singularities associated with the atrial arrhythmia, wherein each of the stages includes a plurality of pulses.

1. (canceled)

2. A method, comprising:
providing a lead configured for implantation in a patient, the lead including:
a proximal portion configured to be connectable to an implantable therapy generator;
a body portion having at least two electrodes; and
a distal portion having at least two electrodes; and
implanting the lead in the patient such that the at least two electrodes on the body portion are positioned at a first heart location of the patient, and such that the at least two electrodes on the distal portion are positioned at a second heart location of the patient; and
causing the implantable therapy generator to deliver a multi-stage therapy according to a program, wherein various combinations of at least one electrode of the body portion of the lead and at least one electrode of the distal portion of the lead are activated in response to an indication an arrhythmia has occurred, the multi-stage therapy including at least a first stage for unpinning of one or more singularities associated with the arrhythmia, a second stage for anti-repinning of the one or more singularities associated with the arrhythmia, and a third stage for extinguishing of the one or more singularities associated with the arrhythmia, wherein each of the stages includes a plurality of pulses.

3. The method according to claim 2, further comprising selecting the various combinations of electrodes prior to, during, or after implantation of the lead.

4. The method according to claim 2, further comprising causing the implantable therapy generator to deliver a multi-stage therapy in response to detection of a location of a reentrant cardiac arrhythmia, the therapy being configured to activate at least two pairs of electrodes, and wherein each pair of electrodes includes at least one electrode on the body portion of the lead and at least one electrode on the distal portion of the lead.

5. The method according to claim 2, wherein each stage of the multi-stage therapy includes a duration, and wherein each therapy stage duration is dependent upon the duration of the arrhythmia.

6. The method according to claim 2, wherein each stage of the multi-stage therapy includes a duration, and wherein the duration of each successive therapy stage is longer than the duration of the previous stage.

7. The method according to claim 2, further comprising delivering an additional therapy subsequent to the multi-stage therapy, the additional therapy being programmed to activate one electrode on the distal portion of the lead to provide cardiac pacing.

8. The method according to claim 2, wherein delivery of the multi-stage therapy is configured to maximize current density between two or more electrodes.

9. The method according to claim 2, wherein delivery of the multi-stage therapy is configured to modify a refractory state at one or more heart locations.

10. The method according to claim 9, wherein delivery of the multi-stage therapy is configured to prolong the refractory state.

11. The method according to claim 2, wherein delivery of the multi-stage therapy is configured to increase defibrillatory efficacy.

12. The method according to claim 2, wherein the implantable therapy generator is further configured to search through numerous electrode combinations to identify one or more arrhythmias.

13. The method according to claim 12, wherein the implantable therapy generator learns which electrode combinations establish each of the one or more arrhythmias, and the implantable therapy generator is configured to deliver a multi-stage therapy that maximizes termination with minimal energy of at least one of the one or more arrhythmias using the learned electrode combinations.

14. The method according to claim 2, further comprising causing the implantable therapy generator to deliver a more aggressive multi-stage therapy in response to the detection of an arrhythmia of longer duration and/or greater maturity.

15. The method according to claim 2, wherein the second heart location comprises a location different from the first heart location.

16. The method according to claim 2, wherein the first heart location comprises a location within the heart, and the second heart location comprises a location within a blood vessel proximate the heart.

17. The method according to claim 2, wherein the lead is oriented such that the proximal portion, the body portion, and the distal portion at least partially surround the heart.
